(19) United States
(12) Patent Application Publication
(10) Pub. No.: US 2024/0206547 A1
(43) Pub. Date: Jun. 27, 2024

(54) Aerosol Generation Device with Manually Adjustable Resistance to Draw

(71) Applicant: JT International SA, Geneva (CH)

(72) Inventors: Jaakko Mcevoy, Vienna (AT); Christoph Lungenschmied, Vienna (AT)

(73) Assignee: JT International SA, Geneva (CH)

(21) Appl. No.: 18/289,066

(22) PCT Filed: Apr. 27, 2022
(86) PCT No.: PCT/EP2022/061178

§ 371 Date: Oct. 31, 2023

(30) Foreign Application Priority Data
May 10, 2021 (EP) 21173087.4

(51) Int. Cl. A24F 40/48; A24F 40/20; A24F 40/51; A24F 40/60

(52) U.S. Cl. CPC A24F 40/48; A24F 40/20; A24F 40/51; A24F 40/60

(57) ABSTRACT

An aerosol generation device includes
an aerosol generation unit arranged for transforming an aerosol-forming substance mixed with air into an aerosol that may be inhaled by a user through successive draws;
   an air flow channel having an inner diameter, allowing air, sucked by a user, to pass through and to be mixed with the aerosol-forming substance, and having a deformable section that is deformable to change its inner diameter, and
a manual adjusting mechanism arranged for acting on this deformable section to induce its deformation when actuated by the user.

### FIELD OF THE INVENTION

The present invention relates to an aerosol generation device, and more precisely to the adjustment of the resistance to draw of such a device.

### BACKGROUND

Some aerosol generation devices comprise an aerosol generation unit arranged for transforming an aerosol-forming substance mixed with air into an aerosol that may be inhaled by a user through successive draws (or puffs or else inhalation phases), and an air flow channel allowing air, sucked by this user, to pass through and to be mixed with this aerosol-forming substance.

When this type of aerosol generation device is portable, i.e. usable when held by a user, it further comprises a battery (or power source) possibly rechargeable and storing electrical energy that is used by the aerosol generation unit for generating the aerosol. In this case the aerosol generation device may be a vaporizer or an electronic cigarette.

In the following description the term “aerosol-forming substance” is used to designate any substance that is aerosolizable in air to form an aerosol. It may, for instance, be in liquid form, in solid form, or in a semi liquid form. So, it may be a liquid, gel, paste or wax or the like, or any combination of these.

The aerosol-forming substance may comprise one or more of nicotine, polyol, caffeine or other active components. An active component may be carried by a carrier which may include propylene glycol or glycerin, for instance. A flavoring may also be present in the aerosol-forming substance. This flavoring may include Ethylvanillin (vanilla), menthol, Isoamyl acetate (banana oil) or similar, for instance.

Moreover, in the following description the term “aerosol” may include a suspension of substance as one or more of solid particles, liquid droplets and gas. Such a suspension may be in a gas including air. Aerosol herein may generally refer to, or include, a vapor, and may include one or more components of the aerosol-forming substance.

Actually, most of the aerosol generation devices are configured to offer a constant and mean resistance to draw. So, for some users the resistance to draw is too weak while for some other users the resistance to draw is too strong. Moreover, this prevents some users to change the resistance to draw during a vaping session or between successive vaping sessions.

It has been proposed in the patent document US-A1 2021/0015158 to use an air flow channel having a deformable section around which permanent magnets are set. So, when a magnetic field is generated by a dedicated device, it causes these permanent magnets to press the deformable section and therefore to reduce its inner diameter, which increases the resistance to draw of the aerosol generation device. Such a solution increases notably the weight, the complexity and the cost of the aerosol generation device, but also the number of points of failure in the operation loop, which shortens the lifetime. Moreover, this solution is not fully user controlled because the modification of the resistance to draw is induced by the electromagnetic interaction between the generated magnetic field and the permanent magnets.

Therefore, an object of this invention is to improve the situation, and notably to reduce the number of points of failure in the operation loop to make the aerosol generation device more robust and longer lasting.

### SUMMARY OF THE INVENTION

The proposed invention provides notably an embodiment of an aerosol generation device comprising an aerosol generation unit arranged for transforming an aerosol-forming substance mixed with air into an aerosol that may be inhaled by a user through successive draws, and an air flow channel allowing air, sucked by this user, to pass through and to be mixed with this aerosol-forming substance, and having an inner diameter.

This aerosol generation device is characterized in that a wall of its air flow channel has a deformable section that is deformable to change the inner diameter of the air flow channel so as to adjust a resistance to draw through the air flow channel, and in that it further comprises a manual adjusting means arranged for acting on this deformable section to induce its deformation when it is manually actuated by the user wishing to adjust said resistance to draw.

This allows the user to adjust manually and in a very simple manner the inner diameter of the air flow channel and therefore the resistance to draw of his aerosol generation device.

The embodiment of aerosol generation device may comprise other aspects or features, considered separately or combined, as defined hereafter:
the manual adjusting means may be a screw or a mechanical mechanism. When the manual adjusting means is a mechanical mechanism, it may be chosen from a group comprising a lever, a scotch yoke mechanism, and a Scott Russell mechanism, for instance;
it may also comprise at least one strain gauge sensor arranged for measuring a deformation of the deformable section, and a controller arranged for determining a degree of restriction of the deformable section based on the measured deformation and for controlling heater operation of the aerosol generation unit according to this determined degree of restriction;
said airflow channel may include a chamber operable to receive said aerosol-forming substance. The chamber may be a heating chamber.
the aerosol generation device may comprise a heater, which may, for example, be located in or adjacent to the heating chamber (if present) to provide heating to an aerosol-forming substrate located within the heating chamber.
changing said inner diameter may be operable to reduce a cross-sectional area of the air flow channel. Thus the resistance to draw through the air flow channel may be adjusted by changing the cross sectional area of the air flow channel using the deformable section.
the deformable section may be deformable inwardly towards a central axis of the air flow channel. The deformable section may comprise a flexible membrane. For instance, the flexible membrane may be made from a silicone elastomer or a flexible polymer;
in a variant of embodiment, the deformable section may comprise a wall section movable to protrude into the air flow channel;
in a first example of embodiment, the aerosol generation unit may comprise an area intended for containing the aerosol-forming substance in solid form, and the deformable section may comprise at least a part of this area to compress the aerosol-forming substance when it is deformed;
in a second example of embodiment, the deformable section may be comprised in a part of the air flow channel that is located upstream of the aerosol generation unit and arranged for providing the latter with air originating from outside;
the aerosol-forming substance may comprise a tobacco material or at least one polyol;
the aerosol generation device may also comprise a rechargeable power source storing electrical energy;
the aerosol generation device may constitute an electronic cigarette.

### BRIEF DESCRIPTION OF THE FIGURES

The invention and its advantages will be better understood upon reading the following detailed description, which is given solely by way of non-limiting examples and which is made with reference to the appended drawings, in which:

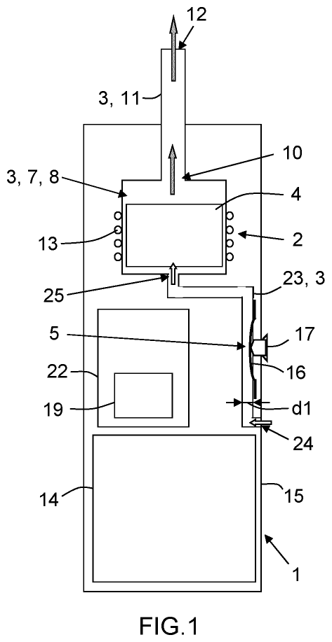

the FIG. 1 schematically and functionally illustrates a first example of embodiment of an aerosol generation device according to the invention,

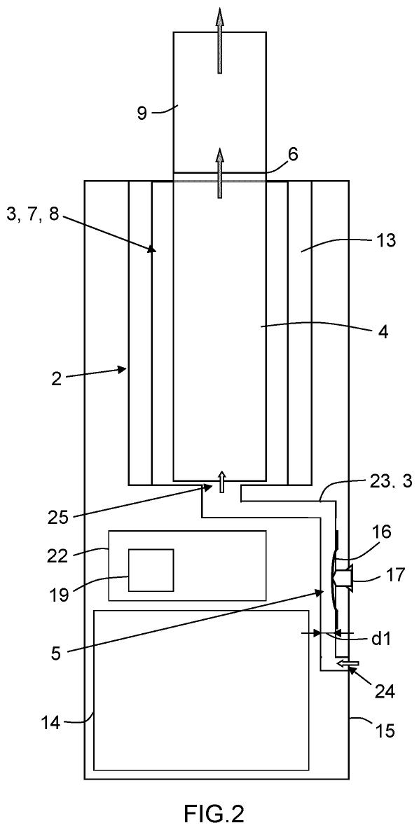

the FIG. 2 schematically and functionally illustrates a second example of embodiment of an aerosol generation device according to the invention,

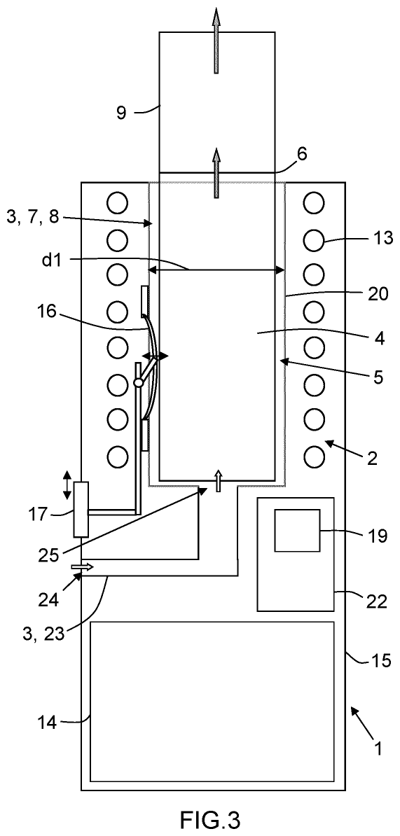

the FIG. 3 schematically and functionally illustrates a third example of embodiment of an aerosol generation device according to the invention,

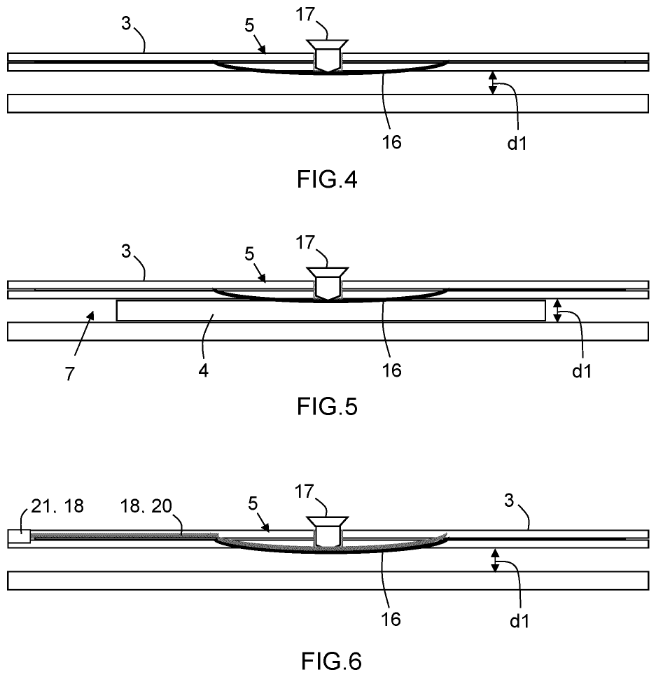

the FIG. 4 schematically and functionally illustrates a first example of air flow channel comprising a deformable section,

the FIG. 5 schematically and functionally illustrates a second example of air flow channel comprising a deformable section containing an aerosol-forming substance, and

the FIG. 6 schematically and functionally illustrates a third example of air flow channel comprising a deformable section with a strain gauge sensor.

### DETAILED DESCRIPTION OF EMBODIMENTS

The invention aims, notably, at offering an aerosol generation device 1 allowing its user to adjust manually and in a simple manner the resistance to draw.

In the following description it will be considered that the aerosol generation device 1 is (or constitutes) an electronic cigarette (or e-cigarette or else personal vaporizer). But an aerosol generation device 1 according to the invention could be of another type, as soon as it is arranged for transforming an aerosol-forming substance mixed with air into an aerosol (possibly close to room temperature) that may be inhaled by a user through successive puffs (or draws or inhalation phases) during a vaping session. More generally, the invention concerns any type of aerosol generation device, and notably the so-called “E-vapor devices” comprising a resistive or inductive heater for heating a liquid and “T-vapor (or heat-not-burn (or “HnB”)) devices” comprising a heater for heating a “solid” substance (for instance a tobacco stick) and similar to a traditional cigarette.

It is recalled that an “aerosol-forming substance” is used to designate any material that is aerosolizable in air to form an aerosol. It may, for instance, be in liquid form, in solid form, or in a semi liquid form. So, it may be a liquid, gel, paste or wax or the like, or any combination of these, and may comprise one or more of nicotine, polyol, caffeine or other active components, or else flavoring. For instance, the aerosol-forming substance may comprise a tobacco material.

In the following description, the aerosol generation device 1 is considered to be an electronic cigarette, and it will be considered that the aerosol-forming substance is in a liquid form (or state). But, as mentioned above the invention concerns any type of aerosol generation device.

It is also recalled that the term “aerosol” may include a suspension of substance as one or more of solid (very small) particles, liquid droplets and gas, and that such a suspension may be in a gas including air.

As illustrated in FIGS. 1 to 3, an aerosol generation device 1, according to the invention, comprises at least an aerosol generation unit 2, an air flow channel 3, and a manual adjusting means 17.

The aerosol generation unit 2 is arranged for transforming an aerosol-forming substance 4 mixed with air into an aerosol that may be inhaled by a user through successive draws (or puffs or inhalation phases) during a vaping session.

The air flow channel 3 allows the air, sucked by the user, to pass through and to be mixed with the aerosol-forming substance 4. This air flow channel 3 has a deformable section 5 that is deformable to change its inner diameter d1.

For instance, the aerosol-forming substance 4 may be contained in a consumable 6 that must be manually replaced by the user when there is no more aerosol-forming substance 4 in it. This consumable 6 can be installed manually, at least partly, into a dedicated cavity of the aerosol generation unit 2. For instance, and as illustrated in FIGS. 2 and 3, this dedicated cavity can be defined by a part 7 of the air flow channel 3 which may be, also (but not necessarily), a heating chamber 8 of the aerosol generation unit 2. In this case, the aerosol-forming substance 4 is fully housed in the heating chamber 8, as illustrated.

Also as illustrated in the non-limiting examples of FIGS. 2 and 3 (where the aerosol generation device 1 is a T-vapor device), the consumable 6 may also comprise a filter 9, downstream of the aerosol-forming substance 4. As for a cigarette, the filter 9 may be partly and temporarily located in the mouth of the user and in this case it is used as a mouthpiece for inhaling the generated aerosol during each puff (or draw).

But, as illustrated in the non-limiting example of FIG. 1 (where the aerosol generation device 1 is one example of an E-vapor device chosen amongst several ones), the heating chamber 8 may comprise an outlet 10 feeding an ending part 11 of the air flow channel 3 with the generated aerosol and comprising another outlet 12 for delivering this aerosol to the user when he is inhaling. In this example the ending part 11 is partly located outside the aerosol generation device 1 and its outlet 12 is temporarily located in the mouth of the user and used as a mouthpiece for inhaling the generated aerosol during each puff (or draw). But this ending part 11 could be connected to a mouthpiece. It is important to notice that an E-vapor device does not comprise necessarily a heating chamber. For instance, the heater may be located inside the e-liquid capsule or outside the e-liquid capsule (without need of a heating chamber).

The aerosol-forming substance 4 is arranged for generating an aerosol when it is heated (without burning) and mixed with air. This heating is performed by a heater 13 supplied with electrical energy originating from a power source 14. This heater 13 belongs to the aerosol generation unit 2.

In the non-limiting examples illustrated in FIGS. 1 to 3, the heater 13 surrounds the heating chamber 8 and therefore the aerosol-forming substance 4. For instance, and as illustrated in the non-limiting example of FIG. 2, the heater 13 may be a thin film heater wrapped around the outer surface of the heating chamber 8 (and therefore of the part 7 of the air flow channel 3) to heat its side walls and at least a part of its internal volume. But in variants illustrated in the non-limiting examples of FIGS. 1 and 3, the heater 13 may be a coil associated with a susceptor or a resistive coil heater. In the first alternative, the coil is arranged for generating an electromagnetic field when it is supplied with an electrical current, and the susceptor is arranged for transforming this electromagnetic field into heat. This susceptor may be a foil of a consumable 6 that surrounds or is located inside the aerosol-forming substance 4.

For instance, the heater 13 may heat the aerosol-forming substance 4 to a temperature comprised between 150° C. and 350° C.

The power source 14 is housed in a body 15 of the aerosol generation device 1. In the non-limiting examples illustrated in FIGS. 1 to 3, this body 15 comprises also the aerosol generation unit 2. But this is not mandatory. Indeed, the aerosol generation unit 2 could be housed in another body coupled to the body 15 by screwing by means of two corresponding threaded portions, or by clipping or else by means of magnets, for instance.

Also for instance, the power source 14 may be a rechargeable battery. In this case the body 15 may comprise an electrical connector to which a charger cable may be connected during a charging session of the rechargeable battery 14. Such a charger cable may be coupled to an (AC) adapter or to a wall socket. The charger cable and/or the (AC) adapter may belong to the aerosol generation device 1.

Also for instance, and as illustrated in the non-limiting examples of FIGS. 1 to 6, the deformable section 5 of the air flow channel 3 may comprise a flexible membrane 16 that is deformable inwardly towards a central axis of this air flow channel 3. This flexible membrane 16 may be an insert that is sealingly fitted in an opening of the air flow channel 3. For instance, this flexible membrane 16 may be made from a silicone elastomer or a flexible polymer.

But in a variant not illustrated, the deformable section 5 could comprise a wall section movable to protrude into the air flow channel. In this case the wall section can be moved perpendicularly to the central axis of the air flow channel 3, preferably sealingly. In this embodiment the wall section may be a solid component, similar to the ones found in laboratory optical mounts.

The manual adjusting means 17 is arranged for acting on the deformable section 5 to induce its deformation when it is manually actuated by a user wishing to adjust the resistance to draw.

This allows the user to adjust manually and in a very simple manner the inner diameter d1 of the air flow channel 3 and therefore the resistance to draw of his aerosol generation device 1. Such a manual adjustment of the inner diameter d1 of the deformable section 5 has the benefit of being fully user controlled, and also low cost. Moreover, it allows reducing the number of points of failure in the operation loop, which makes the aerosol generation device 1 more robust and longer lasting.

For instance, the manual adjusting means 17 may be a screw or a mechanical mechanism.

When the manual adjusting means 17 is a screw, as illustrated in the non-limiting examples of FIGS. 1, 2 and 4 to 6, the screw may be a grub screw, for instance. When the user screws on the screw 17 in a threaded hole defined in a wall of the deformable section 5 and/or body 15, the end of the screw 17 pushes on the flexible membrane 16 (or on the movable wall section), which induces a reduction of the inner diameter d1 of the deformable section 5 and therefore an increase of the resistance to draw of the aerosol generation device 1. Now, when the user unscrews the screw 17, the end of the screw 17 pushes less and less on the flexible membrane 16 (or on the movable wall section), which induces an increase of the inner diameter d1 of the deformable section 5 and therefore a decrease of the resistance to draw of the aerosol generation device 1.

When the manual adjusting means 17 is a mechanical mechanism, it may be a lever (as illustrated in the non-limiting example of FIG. 3), or a scotch yoke mechanism, or else a Scott Russell mechanism, for instance. In this case the mechanical mechanism 17 comprises a sliding member that can be moved translationally on an external face of the body 15 and acting on a lever to move its end perpendicularly to the axis of the air flow channel 3. When the user pushes forward on the sliding member of the mechanical mechanism 17, the lever end moves inwardly towards the central axis of the air flow channel 3 and therefore pushes on the flexible membrane 16 (or on the movable wall section), which induces a reduction of the inner diameter d1 of the deformable section 5 and therefore an increase of the resistance to draw of the aerosol generation device 1. Now, when the user pushes backwards on the sliding member of the mechanical mechanism 17, the lever end moves away from the central axis of the air flow channel 3 and therefore pushes less and less on the flexible membrane 16 (or on the movable wall section), which induces an increase of the inner diameter d1 of the deformable section 5 and therefore a decrease of the resistance to draw of the aerosol generation device 1.

Also for instance, and as partly illustrated in the non-limiting example of FIG. 6 the aerosol generation device 1 may also comprise at least one strain gauge sensor 18 and a controller 19. The (each) strain gauge sensor 18 is arranged for measuring a deformation of the deformable section 5. The controller 19 is arranged for determining a degree of restriction of the deformable section 5 based on this measured deformation and for controlling heater operation of the aerosol generation unit 2 according to this determined degree of restriction. This degree of restriction can influence the puff volume, and therefore the heater needs to be controlled accordingly.

As an example, for low resistance to draw (no blockage) the user can easily draw a large amount of fresh air through the device and therefore the heater (or coil) may need slightly more power to give the same/desired vapor density (cloud) as if the user had set a high resistance to draw, and he finds it hard to pull the same large amount of air though at the same speed. In this case the heater (or coil) may need slightly less power to ensure no over saturation of the vapor/air flow and lead to a possible increased condensation.

For instance, the strain gauge sensor 18 may comprise a strain gauge circuit 20 at least partly in contact with the flexible membrane 16 (or on the movable wall section) by a suitable adhesive, such as cyanoacrylate, and a microcontroller 21. The strain gauge circuit 20 may comprise a metallic foil pattern which is deformed with the deformable section 5, causing its electrical resistance to change. The resistance change can be measured by a Wheatstone bridge coupled to the microcontroller 21 which transforms the measured resistance change into a measured deformation that it transmits to the controller 19.

The controller (or control unit) 19 may comprise at least a processor and a memory arranged for performing operations for controlling the aerosol generation unit 2 (and notably its heater 13) during a vaping session and also the power source 14 during a possible charging session.

For instance, the processor may be a digital signal processor (or DSP), or an application specific integrated circuit (ASIC), or else a field programmable gate array (FPGA). More generally, the processor may comprise integrated (or printed) circuits, or several integrated (or printed) circuits connected therebetween through wired or wireless connections. The term “integrated (or printed) circuits” refers here to any type of device capable of carrying out at least one electric or electronic operation.

Also for instance, the memory may be a random access memory (or RAM). But it may be any type of device arranged for storing program instructions for the processor.

Generally speaking, the functions of the controller (or control unit) 19 may be carried out through the operation of program logic, through dedicated logic, through the interaction of program control and dedicated logic, or even manually (by the user). These functions may be provided through the use of dedicated hardware as well as hardware capable of executing software in association with appropriate software.

As illustrated in the non-limiting example of FIG. 1, the controller (or control unit) 19 (and notably its processor and memory) may be fixed onto a printed circuit board (or PCB) 22 (here housed in the body 15).

The controller (or control unit) 19 may also comprise, in addition to its processor and memory, an input interface, notably for receiving the signals delivered by the possible microcontroller 21, for using them in calculus and processing, possibly after having processed and/or demodulated and/or amplified them, in a manner known by those skilled in the art, by means of an additional digital signal processor. The controller (or control unit) 19 may also comprise a mass memory, notably for storing intermediate data produced during its calculus and processing, and an output interface for delivering messages and instructions at least for controlling the aerosol generation unit 2 (and notably its heater 13) and the electronic component(s) (such as switch(es)) supplying the electrical power (stored in the power source 14) to the aerosol generation unit 2.

As illustrated in the non-limiting examples of FIGS. 3 and 5, the aerosol generation unit 2 may comprise an area that is intended for containing the aerosol-forming substance 4 in solid form (possibly inside a consumable 6). This area is the above mentioned part 7 of the aerosol generation unit 2. In this case, the deformable section 5 may comprise at least a part of this area 7 to compress the aerosol-forming substance 4 when it is deformed (by the manual adjusting means 17). Indeed, the flexible membrane 16 (or the movable wall section) is located next to the area 7 and therefore next to the aerosol-forming substance 4, so that when the flexible membrane 16 (or on the movable wall section) protrudes inwardly into the area 7, it causes compression of the aerosol-forming substance 4 and then an increase of the resistance to draw. In such an embodiment, the air flow channel 3 comprises also a section 23 that is located upstream of the aerosol generation unit 2 and arranged for providing the latter (2) with air originating from outside. More precisely, this section 23 comprises an air inlet 24 communicating with the outside (and defined in the body 15), and an air outlet 25 communicating with the heating chamber 8.

For instance, and as illustrated in the non-limiting examples of FIGS. 1, 2 and 4 to 6, the deformable section 5 may be comprised in the part (or section) 23 of the air flow channel 3 that is located upstream of the aerosol generation unit 2 and arranged for providing the latter (2) with air originating from outside (through the air inlet 24 and air outlet 25).

It should be appreciated by those skilled in the art that some block diagrams of FIGS. 1 to 6 herein represent conceptual views of illustrative circuitry embodying the principles of the invention.

The description and drawing merely illustrate the principles of the invention. It will thus be appreciated that those skilled in the art will be able to devise various arrangements that, although not explicitly described or shown herein, embody the principles of the invention and are included within its spirit and scope. Furthermore, all examples recited herein are principally intended expressly to be only for pedagogical purposes to aid the reader in understanding the principles of the invention and the concepts contributed by the inventor(s) to furthering the art, and are to be construed as being without limitation to such specifically recited examples and conditions. Moreover, all statements herein reciting principles, aspects, and embodiments of the invention, as well as specific examples thereof, are intended to encompass equivalents thereof.

1. An aerosol generation device, comprising:
i) an aerosol generation unit arranged for transforming an aerosol-forming substance mixed with air into an aerosol that may be inhaled by a user through successive draws, and
ii) an air flow channel allowing air, sucked by said user, to pass through and to be mixed with said aerosol-forming substance, and having an inner diameter,
wherein a wall of said air flow channel has a deformable section that is deformable to change said inner diameter so as to adjust a resistance to draw through the air flow channel, and
wherein said aerosol generation device further comprises a manual adjusting device arranged for acting on said deformable section to induce deformation of said deformable section when said manual adjusting device is manually actuated by said user.

2. The aerosol generation device according to claim 1, wherein said manual adjusting device is a screw or a mechanical mechanism.

3. The aerosol generation device according to claim 2, wherein said mechanical mechanism is a lever, a scotch yoke mechanism, or a Scott Russell mechanism.

4. The aerosol generation device according to claim 1, further comprising at least one strain gauge sensor arranged for measuring a deformation of said deformable section, and a controller arranged for determining a degree of restriction of said deformable section based on said measured deformation and for controlling heater operation of said aerosol generation unit according to said determined degree of restriction.

5. The aerosol generation device according to claim 1, wherein said airflow channel includes a chamber operable to receive said aerosol-forming substance.

6. The aerosol generation device according to claim 1, wherein said aerosol generation device comprises a heater.

7. The aerosol generation device according to claim 1, wherein said deformable section that is deformable to change said inner diameter is operable to adjust a cross-sectional area of the air flow channel.

8. The aerosol generation device according to claim 1, wherein said deformable section is deformable inwardly towards a central axis of said air flow channel.

9. The aerosol generation device according to claim 1, wherein said deformable section comprises a flexible membrane.

10. The aerosol generation device according to claim 9, wherein said flexible membrane includes a silicone elastomer or a flexible polymer.

11. The aerosol generation device according to claim 1, wherein said deformable section comprises a wall section movable to protrude into said air flow channel.

12. The aerosol generation device according to claim 1, wherein said aerosol generation unit comprises an area arranged for containing said aerosol-forming substance in solid form, and said deformable section comprises at least a part of said area to compress said aerosol-forming substance when it is deformed.

13. The aerosol generation device according to claim 1, wherein said deformable section is comprised in a part of said air flow channel that is located upstream of said aerosol-forming substrate, said part arranged for providing said aerosol generation unit with air originating from outside.

14. The aerosol generation device according to claim 1, wherein said aerosol generation device is an electronic cigarette.
